(19) United States
(12) Patent Application Publication
(10) Pub. No.: US 2026/0083933 A1
(43) Pub. Date: Mar. 26, 2026

(54) RELAXATION SYSTEM WITH SCENT RELEASE

(71) Applicant: Calm and Sense Technologies, LLC, Palm Beach Gardens, FL (US)

(72) Inventors: Daniel Shuter, New York, NY (US); Angela Sun, New York, NY (US); David Shuter, Palm Beach Gardens, FL (US)

(21) Appl. No.: 19/367,950

(22) Filed: Oct. 24, 2025

Related U.S. Application Data
(63) Continuation-in-part of application No. 18/895,816, filed on Sep. 25, 2024, now Pat. No. 12,485,249.

(51) Int. Cl. A61M 21/02; A61M 21/00

(52) U.S. Cl. CPC A61M 21/02; A61M 2021/0016; A61M 2210/10

(57) ABSTRACT

A relaxation device includes an enclosure, a vibrator, a scent release mechanism, at least one processing device, and a power source. The vibrator is provided in the enclosure and is configured to induce vibrations of at least a portion of the enclosure. The scent release mechanism is at least partially mounted within the enclosure. The processing device is in electrical communication with the vibrator and the scent release mechanism and is configured to control operation of the vibrator and the scent release mechanism. The power source is in the enclosure and electrically connected with the processing device, the vibrator and the scent release mechanism.

### BACKGROUND

Various relaxation devices have been developed to help individuals manage stress and promote relaxation. However, many existing solutions are limited in their approach, often focusing on a single sensory experience. There is a need for a more comprehensive relaxation device that can engage multiple senses simultaneously, providing a more effective and personalized relaxation experience

### SUMMARY

In view of the foregoing, a relaxation device includes an enclosure, a vibrator, a scent release mechanism, at least one processing device, and a power source. The vibrator is provided in the enclosure and is configured to induce vibrations of at least a portion of the enclosure. The scent release mechanism is at least partially mounted within the enclosure. The processing device is in electrical communication with the vibrator and the scent release mechanism, and is configured to control operation of the vibrator and the scent release mechanism. The power source is in the enclosure and electrically connected with the processing device, the vibrator and the scent release mechanism.

A scent capsule for use with a relaxation device is also disclosed. The scent capsule includes a capsule housing, a scent solution in the capsule housing and a capillary wick extending from an interior of the capsule housing. The capsule housing has a volume between 0.5 and 3.0 mL. The capillary wick extends through a wick opening in the capsule housing to an exterior of the capsule housing.

A method of operating a relaxation device is also disclosed. The method includes controlling a vibrator disposed in an enclosure to induce vibrations of at least a portion of the enclosure, detecting a person’s cardiac signals via electrodes mounted to the enclosure and at least one processing device in electrical communication with the electrodes, and comparing the detected cardiac signals to a target cardiac signal via the at least one processing device. If the detected cardiac signals correspond to the target cardiac signal, then a scent solution is emitted from the enclosure via a scent release mechanism in electrical communication with the processing device. If the detected cardiac signals do not correspond to the target cardiac signal, then the method returns to detecting the person’s cardiac signals via the electrodes mounted to the enclosure and the processing device in electrical communication with the electrodes.

### BRIEF DESCRIPTION OF THE DRAWINGS

FIG. 1 is a schematic depiction of an enclosure of the relaxation device resting on a user’s sternum.

FIG. 2 is a perspective view of an example of the relaxation device of FIG. 1.

FIG. 3 a cross-sectional view of the relaxation device, showing the arrangement of a vibrator, processing device, and power source within an enclosure.

FIG. 4 is a schematic depiction of a relaxation system including the relaxation device.

FIG. 5 is a close up cross-sectional view of a replaceable scent capsule received in a scent capsule cavity and cooperating with a scent release mechanism of the relaxation device.

FIG. 6 is a perspective view of an alternative scent capsule with a cap removed.

FIG. 7 a cross-sectional view of the alternative scent capsule shown in FIG. 6.

FIG. 8 is a schematic cross-sectional view of the alternative scent capsule shown in FIG. 6.

FIGS. 9 and 10 are perspective views of a hollow body for the alternative scent capsule shown in FIG. 6.

FIG. 11 top view of a disk and atomizer for the scent release mechanism.

FIG. 12 is a flow diagram depicting an example of a method of operating the relaxation system.

### DETAILED DESCRIPTION

The detailed description and specific examples, while describing particular embodiments, are intended for purposes of illustration only and are not intended to limit the scope of the invention. These embodiments and other features, aspects, and advantages will become better understood from the following description, appended claims, and accompanying drawings. The figures are merely schematic and may not be drawn to scale, and the same reference numerals are used throughout the figures to indicate the same or similar parts.

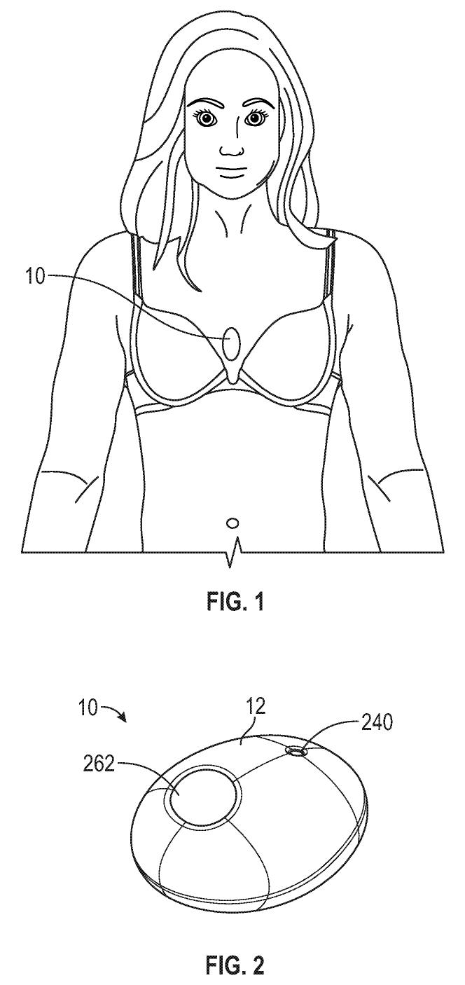

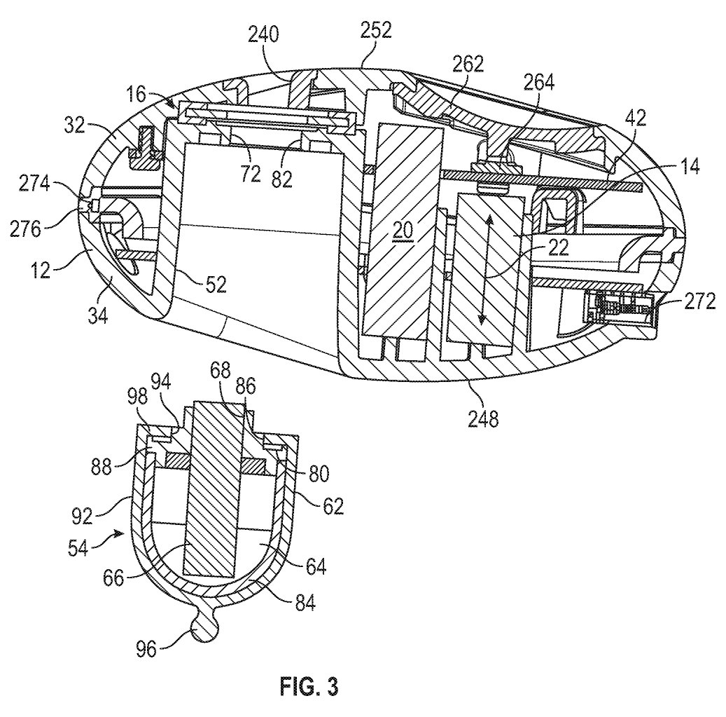

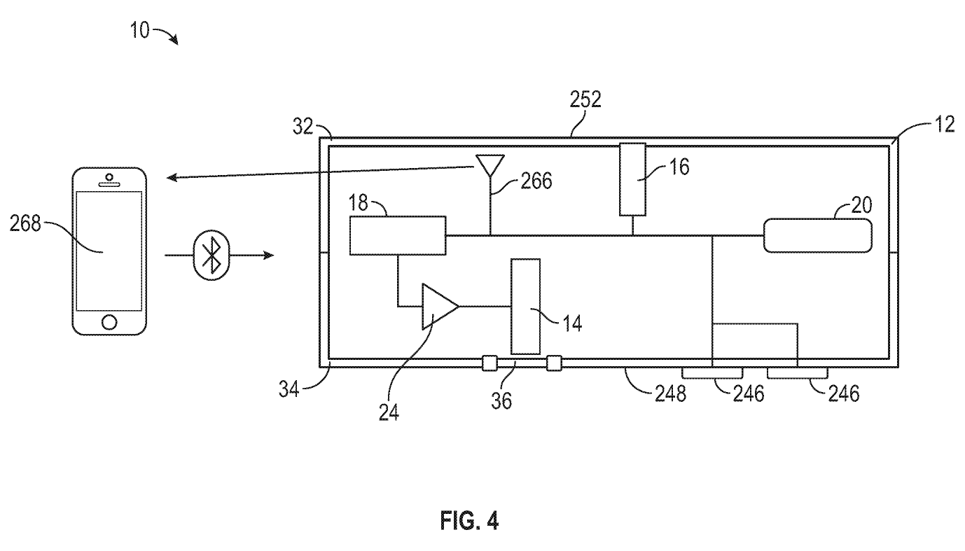

FIG. 1 depicts a relaxation device 10 that is intended to rest on a person’s sternum for guiding a user in paced breathing to encourage relaxation and/or sleep induction. The relaxation device 10 integrates vibration and scent release mechanisms within an enclosure 12. As seen in FIGS. 3 and 4, the relaxation device 10 includes a vibrator 14 in the enclosure 12 and a scent release mechanism 16 at least partially mounted within the enclosure 12. The relaxation device 10 also includes at least one processing device, which can include a processing unit 18 (schematically depicted in FIG. 4) and other components examples of which will be described in more detail below. The at least one processing device controls the operation of both the vibrator 14 and the scent release mechanism 16, allowing for synchronized or independent activation of these features. The relaxation device 10 also includes a power source 20 in the enclosure 12 and electrically connected with the at least one processing device, the vibrator 14 and the scent release mechanism 16.

The vibrator 14 is mounted within the enclosure 12 and is configured to impart a force on at least a portion of the enclosure 12. The vibrator 14 can be designed to produce linear motion substantially along a single axis 22, such as a speaker coil or a linear vibration motor. The vibrator 14 could also be a rotary vibrator, e.g., an eccentric rotating mass (ERM) motor which generates multidirectional vibrations.

In a more particular example, the vibrator 14 can be a linear vibration motor designed to operate within a specific frequency range that can provide effective haptic sensations to the user while remaining relatively quiet to the user. Infrasonic sound, also known as low frequency sound, is a type of sound wave with a frequency below the human hearing range, which is generally 20 Hertz (Hz) or lower. The relaxation device 10 can be configured such that the linear vibration motor operates at frequencies between 45-100 Hz, which is near the infrasonic range. The linear vibration motor can have a resonant frequency between 45-100 Hz. More specifically, the linear vibration motor can have a resonant frequency between 70-90 Hz.

When the vibrator 14 is a linear vibration motor, the linear vibration motor can be designed to provide significant vibration force while maintaining energy efficiency and quiet operation. The linear vibration motor can be configured to generate a root mean square acceleration of at least 0.2 g²/Hz. This measure of vibration intensity ensures that the device provides a noticeable haptic cue to the user while also stimulating the vagus nerve, which runs under the sternum, when the relaxation device 10 is resting on the user’s sternum. The linear vibration motor can be configured to achieve the aforementioned acceleration for every 200mW of power consumed. This high efficiency allows for extended operation times and makes the relaxation device 10 suitable for portable, battery-powered use.

The operation of the vibrator 14 is controlled by the at least one processing device, which can include the processing unit 18 positioned within the enclosure. The at least one processing device can generate a sine wave signal to drive the vibrator 14. The amplitude of the sine wave can be varied over time, allowing for dynamic vibration patterns, e.g., to create pulsing effects or gradually increase or decrease the intensity of the vibration. With reference to FIG. 4, an amplifier 24 may be used to boost the sine wave signal before it reaches the vibrator 14. The amplifier 24 could be an audio amplifier or an operational amplifier, with either fixed or adjustable gain. The use of an amplifier 24 allows the device to achieve higher vibration intensities when desired.

The relaxation device 10 includes an enclosure 12 that can take the form shown in FIG. 2; however, the enclosure 12 can take other forms as well. The enclosure 12 is designed to be compact and portable, allowing users to carry and use the relaxation device 10 in various settings. The enclosure 12 may be made of a suitable material such as plastic or metal that can effectively transmit vibrations while also providing durability and protection for the internal components. The enclosure 12 may be designed in various shapes, such as spherical, cylindrical, or ergonomically contoured to fit comfortably on a user's sternum or against different parts of the body. The specific shape chosen can optimize both the transmission of vibrations and user comfort.

With reference to FIGS. 2 and 3, the enclosure 12 can include an upper shell 32 connected to a lower shell 34 via conventional fastening methods including fasteners, adhesives, welding and the like. If desired, and schematically depicted in FIG. 4, the enclosure 12 can include a movable outer wall portion 36 that is directly influenced by the vibrator 14. In some embodiments, the vibrator 14 can be directly connected to the movable outer wall portion 36. This allows the vibration to be directly transferred to the surface that comes into contact with the user. The movable outer wall portion 36 can be connected to a relatively fixed outer wall portion, which in the embodiment illustrated in FIG. 4 is the lower shell 34, via a resilient ring 38. This resilient ring 38 allows for controlled movement of the movable outer wall portion 36 with respect to the relatively fixed outer wall portion, e.g., the lower shell 34, while maintaining the integrity of the enclosure 12 and preventing ingress of dust or moisture into the interior of the enclosure 12. The movable outer wall portion 36 may be made of a slightly more flexible material than the fixed outer wall portion to enhance vibration transmission. The size and shape of the movable outer wall portion 36 can be optimized to provide an effective contact area for the user while maintaining the desired vibration characteristics. When the vibrator 14 imparts a force to the movable outer wall portion 36, the movable outer wall portion 36 moves relative to the fixed outer wall portion. This movement is typically very small (in the range of micrometers to a few millimeters) while being sufficient to create the desired haptic effect.

The at least one processing device, which can include the processing unit 18 in FIG. 4, is in electrical communication with the vibrator 14 and the scent release mechanism 16. The at least one processing device serves as the central control unit for the relaxation device 10. It may comprise one or more microprocessors, microcontrollers, or other suitable integrated circuits, which can be mounted on a circuit board 42 (FIG. 3), capable of executing instructions stored in a memory component. The memory component, which may be integrated with or separate from the processing unit 18 depicted in FIG. 4, can store firmware, software instructions, and data necessary for the operation of the relaxation device 10.

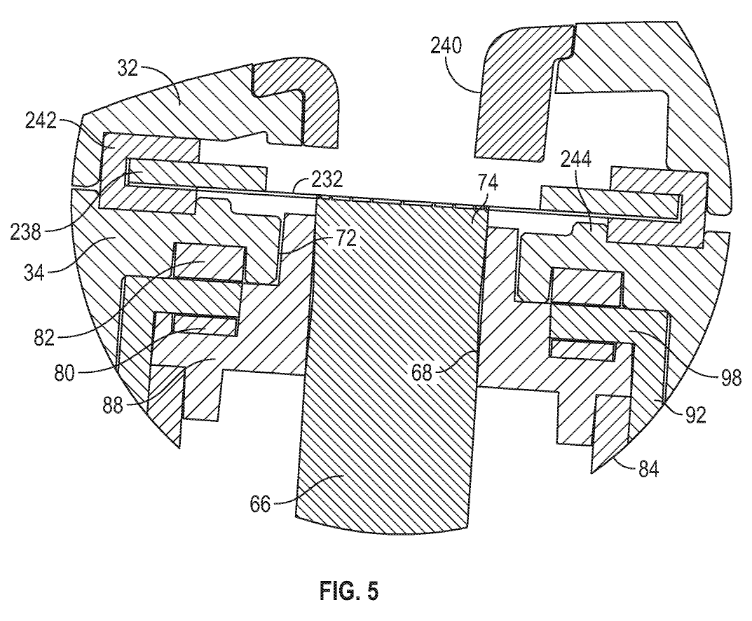

The relaxation device 10 also includes the scent release mechanism 16, which is at least partially mounted within the enclosure 12. As detailed in FIG. 3, the enclosure 12 defines a scent capsule cavity 52 and a replaceable scent capsule 54 is receivable in the scent capsule cavity 52 and operably connectable with the scent release mechanism 16. With reference back to FIG. 3, the replaceable scent capsule 54 includes a capsule housing 62 containing a scent solution 64. A capillary wick 66 extends from an interior of the capsule housing 62 through a wick opening 68 to the exterior of the capsule housing 62. The capsule housing 62 may have a volume between 0.5 and 3.0 mL, and the capillary wick 66 may have a diameter between 3 and 8 mm. FIG. 5 depicts a close up cross-sectional view of the replaceable scent capsule 54 shown in FIG. 3 received in the scent capsule cavity 52 and cooperating with the scent release mechanism 16. The enclosure 12 defines a wick-receiving opening 72 (most clearly seen in FIG. 3) within the scent capsule cavity 52. A free end 74 of the capillary wick 66 is received in or through the wick-receiving opening 72 when the replaceable scent capsule 54 is properly received within the scent capsule cavity 52 for cooperation with the scent release mechanism 16, which is shown in FIG. 5. The free end 74 of the capillary wick 66 does not extend a substantial distance, e.g., less than a few millimeters and preferably less than 2.5 mm, from the capsule housing 62.

To secure the replaceable scent capsule 54 within the scent capsule cavity 52, a magnetic connector 80 is mounted to the capsule housing 62. This magnetic connector 80 cooperates with a magnetic element 82 positioned within the scent capsule cavity 52 of the enclosure 12. The free end 74 of the capillary wick 66 is received in or through the wick-receiving opening 72 when the replaceable scent capsule 54 is received within the scent capsule cavity 52 with the magnetic element 82 cooperating with the magnetic connector 80.

In the embodiment of the replaceable scent capsule 54 depicted in FIG. 3, the capsule housing 62 includes a hollow body 84, which is generally dome shaped. The hollow body 84 includes a plug opening 86 at an end that is inserted furthest into the scent capsule cavity 52. A plug 88 is inserted into the plug opening 86. The plug 88 includes the wick opening 68 and with the capillary wick 66 received in the wick opening 68 closes off the plug opening 86. The magnetic connector 80 is trapped between the plug 88 and a sleeve 92 that covers at least a portion of the hollow body 84 and contacts the plug 88 at a radial shoulder 94. In the embodiment of the replaceable scent capsule 54 depicted in FIG. 3, the sleeve 92 can be made from a more resilient material than the hollow body 84. For example, the sleeve 92 can be made from silicone, and the hollow body 84 can be made from a more rigid plastic or glass, for example. A tab 96 can be formed on the sleeve 92 opposite the end through which the capillary wick 66 extends. With the sleeve 92 being made from a relatively resilient material, if the tab 96 were to contact the user’s skin while in use, there will be relatively little discomfort. By being made from a relatively resilient material, the sleeve 92 provides a gasket 98 covering the magnetic connector 80. The sleeve 92 is shown as covering the entirety of the hollow body 84 in FIG. 3, but it may only cover a portion thereof, while still sealing the plug 88 to the hollow body 84. With the magnetic connector 80 being sandwiched between the gasket 98 of the sleeve 92 and the plug 88, resiliency is provided when the scent capsule 54 is inserted into the scent capsule cavity 52.

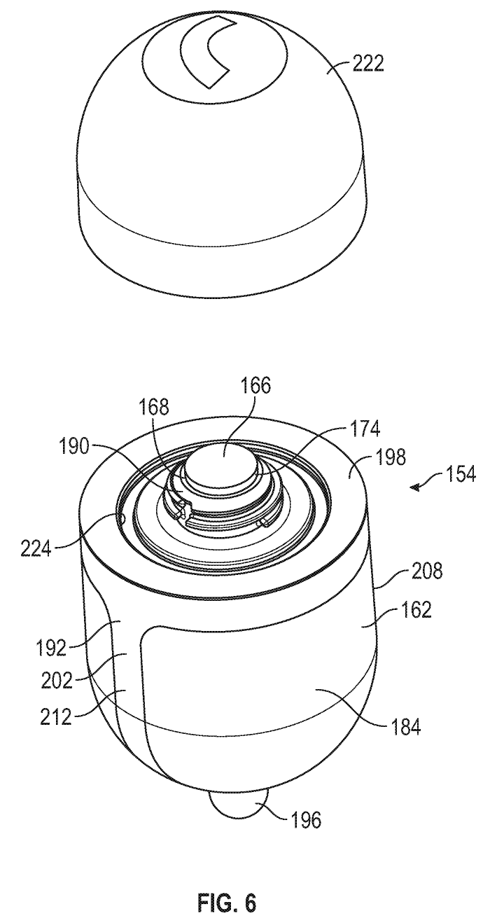

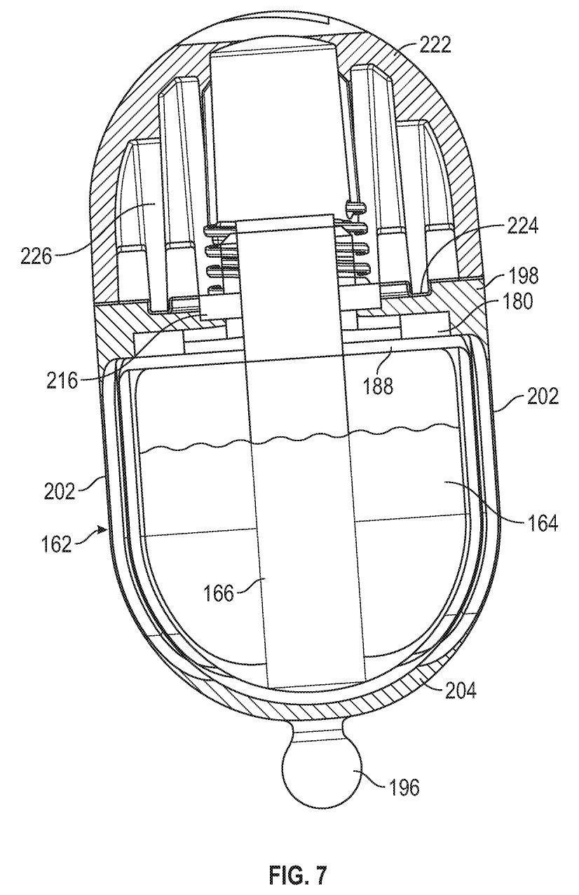

FIGS. 6 - 10 depict another example of an alternative scent capsule 154 that is receivable in the scent capsule cavity 52 (FIG. 3) and operably connectable with the scent release mechanism 16. With reference to FIGS. 6 and 7, the alternative scent capsule 154 includes a capsule housing 162 containing a scent solution 164. A capillary wick 166 extends from an interior of the capsule housing 162 through a wick opening 168 to the exterior of the capsule housing 162. The capsule housing 162 may also have a volume between 0.5 and 3.0 mL, and the capillary wick 166 may have a diameter between 3 and 8 mm. A free end 174 of the capillary wick 166 is received in or through the wick-receiving opening 72 (FIG. 3) when the alternative scent capsule 154 is properly received within the scent capsule cavity 52 for cooperation with the scent release mechanism 16 in a similar manner to that shown in FIG. 5. The free end 174 of the capillary wick 166 does not extend a substantial distance, e.g., less than a few millimeters and preferably less than 2.5 mm, from the capsule housing 162.

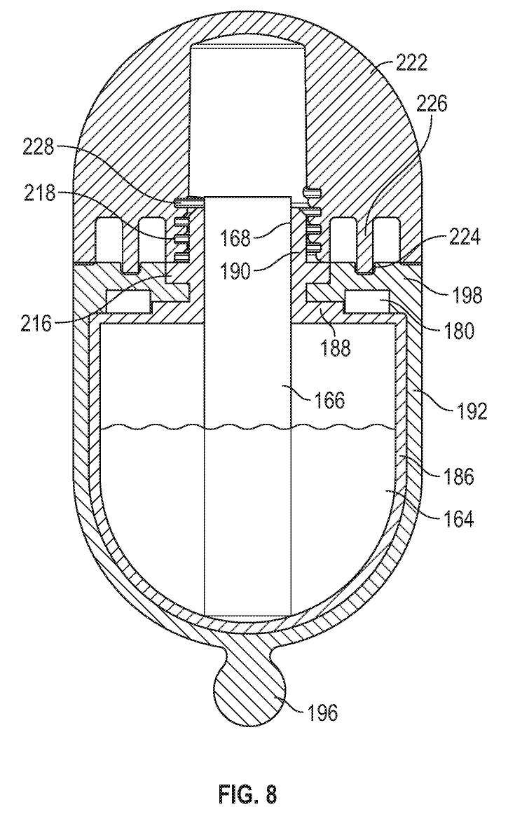

With reference to FIGS. 7 and 8, a magnetic connector 180 is mounted to the capsule housing 162. This magnetic connector 180 cooperates with the magnetic element 82 positioned within the scent capsule cavity 52 of the enclosure 12, which is shown in FIGS. 3 and 5. Similar to that shown in FIGS. 3 and 5, the free end 174 of the capillary wick 166 is received in or through the wick-receiving opening 72 when the alternative scent capsule 154 is received within the scent capsule cavity 52 with the magnetic element 182 cooperating with the magnetic connector 180.

For the alternative scent capsule 154 depicted in FIGS. 6 - 10, the capsule housing 162 includes a hollow body 184, which includes a capsule wall 186 that is generally dome shaped. The hollow body 184 also includes a ledge 188 extending inwardly from the capsule wall 186 and a neck 190 extending away from the ledge 188. The neck 190 defines the wick opening 168. The magnetic connector 180 is trapped between the ledge 188 and a jacket 192 that covers at least a portion of the hollow body 184. The jacket 192 can be made from a more resilient material than the hollow body 84. For example, the jacket 192 can be made from silicone, and the hollow body 184 can be made from a more rigid plastic or glass, for example HDPE or PET. A tab 196 can be formed on the jacket 192 opposite the end through which the capillary wick 166 extends. With jacket 192 being made from a relatively resilient material, if the tab 196 were to contact the user’s skin while in use, there will be relatively little discomfort. By being made from a relatively resilient material, the jacket 192 provides a gasket 198 covering the magnetic connector 180. With the magnetic connector 180 being sandwiched between the gasket 198 provided by the jacket 192 and the ledge 188, resiliency is provided when the alternative scent capsule 154 is inserted into the scent capsule cavity 52 (FIGS. 3 and 5).

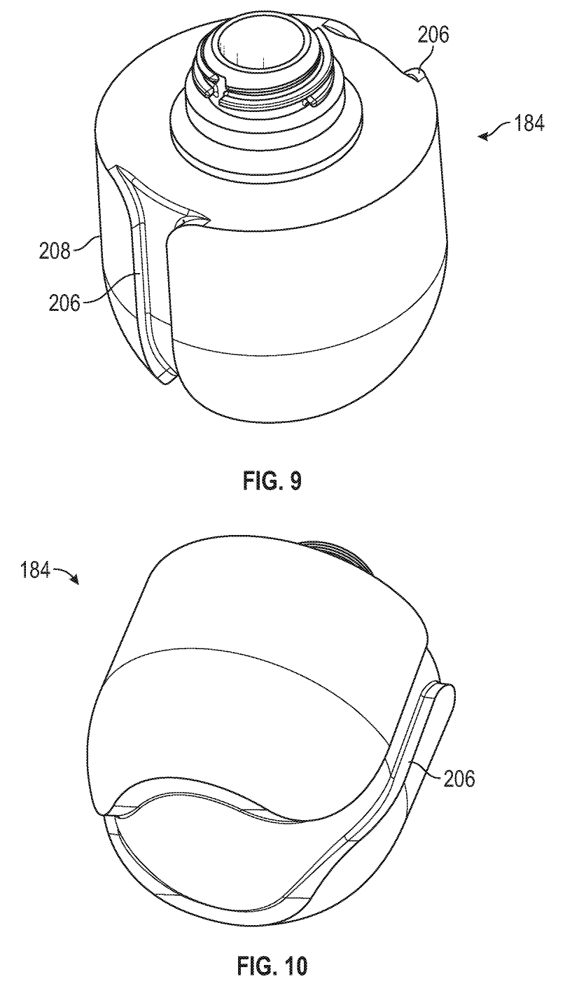

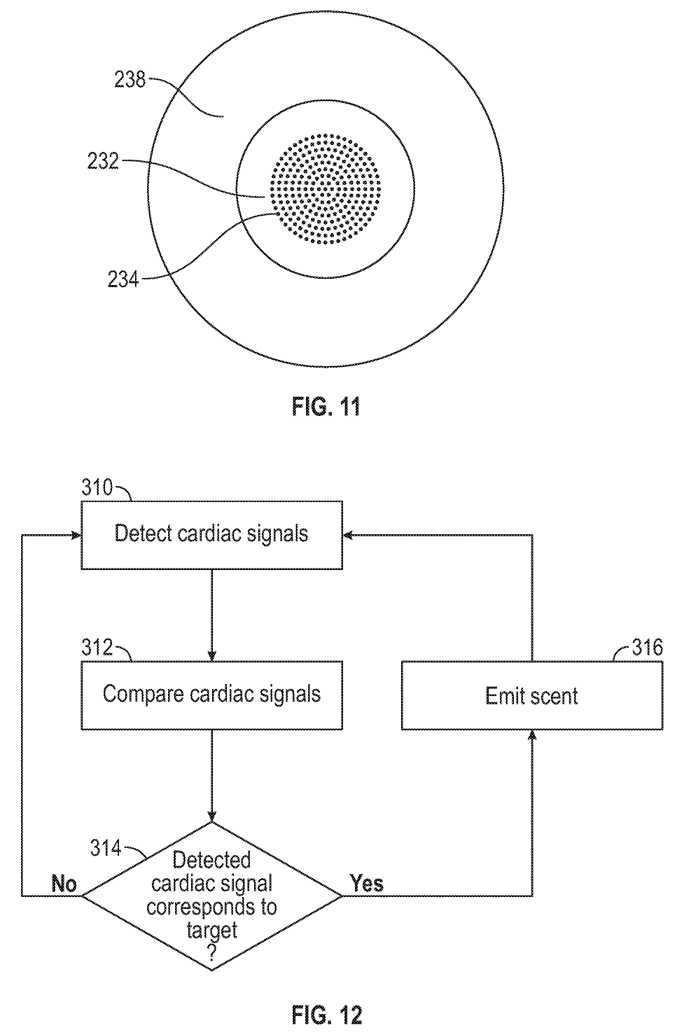

With reference to FIG. 6, the jacket 192 includes at least one band 202 extending from the gasket 198. In the embodiment illustrated in FIGS. 6 - 10, the jacket 192 includes two bands 202 extending from the gasket 198, and the bands 202 are offset 180 degrees from each other with respect to a central axis aligned along a centerline of the capillary wick 166. The jacket 192 further includes a base 204 connected with each band 202 opposite the gasket 198. The base 204 covers a portion of the hollow body 184 located opposite the wick opening 168 and the tab 196 extends away from a portion of the base 204. With reference to FIGS. 9 and 10, which only shows the hollow body 184 prior to application of the jacket 192, the hollow body 184 includes at least one channel 206 extending inwardly from an outer surface 208 of the hollow body 184. With reference also to FIG. 6, each band 202 is received in the channel 206. As seen in FIG. 10, the channel 206 widens at the bottom of the hollow body 184 to accommodate the base 204 of the jacket 192. With reference to FIG. 6, an outermost surface 212 of each band 202 is flush with the outer surface 208 of the hollow body 184. More particularly, the outermost surface 212 of the jacket 192 is flush with the outer surface 208 of the hollow body 184.

The hollow body 184 can be formed via a blow molding process. This allows for the formation of the channel 206, which allows for more volume of scent solution 164 for the alternative scent capsule 154 as compared to the scent capsule 54 depicted in FIGS. 3 and 5. The hollow body 184 can include a flange 216 extending outwardly from the neck 190. The flange 216 can provide a grip location for components used during the blow molding process. External threads 218 can be provided on the neck 190 to allow for a cap 222 to connect with the hollow body 184. With reference back to FIG. 5, the outer thread diameter for the external threads 218 is smaller than the inner diameter of the wick-receiving opening 72 to allow the neck 190 having the external threads 218 to be received in the wick-receiving opening 72 in a manner similar to that shown in FIG. 5.

The jacket 192 can be overmolded onto the hollow body 184. The jacket 192 is shown as an integral unit that includes the gasket 198, the bands 202, the base 204 and the tab 196. If desired, the gasket 198 could be formed separately from the base 204, for example.

With reference again to FIGS. 6 and 8, the gasket 198 includes a gasket channel 224 in which an appendage 226, which can be generally cylindrical and provided in the cap 222, is received when cap 222 is threaded onto the neck 190. Internal threads 228 can be provided in the cap 222 to allow the cap 222 to be threaded on the neck 190. With the cap 222 threaded on the neck 190, evaporation of the scent solution 164 is inhibited. The cap 222 is removed from the neck 190 prior to inserting the alternative scent capsule 154 in the scent capsule cavity 52.

With reference to FIGS. 5 and 11, the scent release mechanism 16 further includes a disk 232 with a plurality of apertures 234. This disk 232 covers the wick-receiving opening 72 within the scent capsule cavity 52. When the replaceable scent capsule 54 or the alternative scent capsule 154 is properly inserted, the free end 74, 174 of the capillary wick 66, 166 is received through the wick-receiving opening 72 and contacts the disk 232, which is shown in FIG. 5 for the replaceable scent capsule 54. As more clearly seen in FIG. 5, however, the capillary wick 66 does not extend a substantial distance, e.g., less than a few millimeters and preferably less than one millimeter, from the plug 88. In a similar manner, the capillary wick 166 does not extend a substantial distance, e.g., less than a few millimeters and preferably less than one millimeter, from the neck 190. Such a construction discourages users from contacting and/or grabbing the capillary wick 66, 166. A piezoelectric atomizer 238 is connected to the disk 232. When energized, the piezoelectric atomizer 238 vibrates the disk 232, drawing the scent solution from the capillary wick 66, 166 and atomizing it as it passes through the apertures 234 in the disk 232. The atomized scent solution then exits the relaxation device 10 through a scent outlet 240 provided in the enclosure 12.

The scent release mechanism 16 depends on capillary action to draw the scent solution 64 from the replaceable scent capsule 54 through the capillary wick 66 to the disk 232. The same is true for the alternative scent capsule 154; the scent release mechanism 16 depends on capillary action to draw the scent solution 164 through the capillary wick 166 to the disk 232. As such, the scent solution 64, 164 is formulated to have a viscosity and specific gravity that is nearer to that of distilled water at 20 degrees C as compared to many oils (e.g., canola oil has a viscosity of around 46 centipoise at 20 degrees C). As such, the scent solution 64, 164 can be formulated to have a viscosity below 10 centipoise at 20 degrees C. In addition or alternatively, the scent solution 64, 164 can be formulated to have a specific gravity within 20% of distilled water at 20 degrees C. Such a formulation can inhibit clogging and enable effective dispersion.

The disk 232 can be made from a durable, non-reactive material that can withstand repeated vibrations and exposure to various scent solutions. Suitable materials may include certain metals (such as stainless steel or titanium) or high-performance polymers that offer chemical resistance and maintain their properties over time. In one embodiment and with reference to FIG. 11, the disk 232 includes at least 50 apertures 234, providing a sufficient number of pathways for the scent solution to be effectively atomized. Each aperture 234 can a maximum diameter between 1 µm and 15 µm. This specific size range is chosen to generate the optimal droplet size for the atomized scent solution, ensuring that it can be easily dispersed into the air and effectively perceived by the user. The apertures 234 can be distributed across the surface of the disk 232 in a pattern designed to promote even and efficient atomization. This distribution may be uniform or may follow a specific pattern optimized for particular design and atomization requirements. The disk 232 is positioned so that it contacts the free end 74, 174 of the capillary wick 66, 166 when the replaceable scent capsule 54 or the alternative scent capsule 154 is properly inserted into the scent capsule cavity 52. This direct contact ensures efficient transfer of the scent solution from the capillary wick 66, 166 for atomization through the apertures 234. The disk 232 is connected to the piezoelectric atomizer 238 in a manner that allows for efficient transfer of vibrational energy as high-frequency vibrations of the disk 232 cause the scent solution to break into fine droplets as it passes through the apertures 234 in route to a scent outlet 240.

The piezoelectric atomizer 238 is designed to operate at a high frequency, typically in the ultrasonic range (>20 kHz). More particularly, the piezoelectric atomizer 238 can operate at frequencies between 50-250 kHz, and most preferably at a resonant frequency of about 113 kHz. This high-frequency provides effective atomization and helps ensure that the relaxation device 10 operates very quietly. A different type of mister is known that uses a piezoelectric atomizer with resonant frequencies around 2MHz, but these are meant to be immersed in the liquid being dispersed and can consume quite a lot of power not making them suitable for use with such a small device. The piezoelectric atomizer 238 is powered by a driver circuit to provide the necessary high-frequency alternating current. This driver circuit is controlled by the at least one processing device and/or the processing unit 18 depicted in FIG. 4 to manage the atomization process.

The piezoelectric atomizer 238 is mounted within the enclosure 12 using a piezoelectric mount 242 made from a dampening material. With reference to FIG. 5, the lower shell 34 can include a locating ridge 244 . The piezoelectric mount 242 is sandwiched between the upper shell 32 and the lower shell 34 of the enclosure 12 in the illustrated embodiment. This mounting arrangement isolates the vibrations of the piezoelectric atomizer 238 from the rest of the relaxation device 10 preventing unwanted noise or interference with other components. The piezoelectric mount 242 being made from a dampening material acts as a gasket, inhibiting the ingress of scent solution into the electronic interior compartment of the enclosure 12 where the vibrator 14, the processing unit 18, and the power source 20 are located. By providing the gasket 98, e.g., the sleeve 92 in FIGS. 3 and 5, at the interface of the scent capsule 54 and the enclosure 12, or lower shell 34, the scent capsule 54 may be able to move, even only slightly, as the disk 232 contacts the capillary wick 66 and the cooperation between magnetic connector 80 and magnetic element 82 can still retain the scent capsule 54 in the scent capsule cavity 52. The same is true for the alternative scent capsule 154; by providing the gasket 198, which is part of the jacket 192 in FIGS. 6 - 10, at the interface of the alternative scent capsule 154 and the enclosure 12, or lower shell 34, the alternative scent capsule 154 may be able to move, even only slightly, as the disk 232 contacts the capillary wick 166 and the cooperation between magnetic connector 180 and magnetic element 82 can still retain the alternative scent capsule 154 in the scent capsule cavity 52.

The relaxation device 10 can also be designed to detect the user's cardiac signals, enabling the relaxation device 10 to adapt its operation based on the user's physiological response. As schematically depicted in FIG. 4, the processing unit 18, which can make up part of at least one processing device, can interface with electrodes 246, which can be dry ECG (electrocardiogram) biopotential electrodes that do not require the typical electrolytic conductive gel and skin preparation as compared to wet electrodes. At least two, and perhaps more, electrodes 246 can be mounted to the enclosure 12 and in electrical communication with the processing unit 18. The electrodes 246 are mounted to the enclosure 12 in a manner such that when the enclosure 12 is placed on the user’s sternum (see FIG. 1) ionic currents from the user’s body surface are converted into electrical signals for later processing by the at least one processing device, which can be useful to adaptively adjust the vibration patterns.

The enclosure 12 includes a lower surface 248, which is depicted in FIGS. 3 and 4, configured to contact a user’s skin and an upper surface 252 opposite and spaced away from the lower surface 248. The electrodes 246 have contact surfaces that are flush with or extend outwardly from an outer surface, and in the illustrated embodiment the lower surface 248, of the enclosure 12. The electrodes 246 are in electrical communication with the processing unit 18 and can be used to measure the user's cardiac signals. The upper surface 252 defines the scent outlet 240 (see FIG. 3) through which atomized scent solution passes.

The at least one processing device, which includes the processing unit 18, is programmed to perform several functions of the relaxation device 10.

The at least one processing device controls the operation of the vibrator 14, which can include adjusting the frequency, intensity, and pattern of vibrations based on predefined settings or user input. The at least one processing device may implement various vibration profiles designed to induce different relaxation states. The at least one processing device manages the operation of the scent release mechanism 16 such as controlling the timing and duration of scent release, as well as potentially adjusting the intensity of scent release by modulating the operation of the piezoelectric atomizer 238.

When the relaxation device 10 is equipped with electrodes 246, the at least one processing device can process and analyze the cardiac signals detected by the electrodes 246. This analysis may involve calculating various cardiac metrics such as heart rate, heart rate variability, or other relevant parameters. Based on the analysis of cardiac signals, the at least one processing device can adapt the operation of the vibrator 14 and scent release mechanism 16. For example, if the detected cardiac signals indicate a high stress level, the at least one processing device might intensify the vibration and scent release to promote relaxation.

The relaxation device 10 includes an on/off button 262 operably connected with an on/off switch 264 that is in electrical communication with the at least one processing device. The on/off switch 264 can be mounted to the circuit board 42 located within the enclosure 12. The on/off button 262 can be offset from the scent outlet 240 along the upper surface 252 of the enclosure 12. Where the relaxation device 10 includes user interface elements such as buttons, touch sensors, or displays, the at least one processing device manages these interfaces, interpreting user inputs and providing appropriate feedback or device responses.

With reference to FIG. 4, wireless communication modules 266 (e.g., Bluetooth Low Energy) can be provided as part of the at least one processing device to provide communication between a smart phone 268. In such an instance, the processor on the smart phone 268 can form a component of the at least one processing device and the smart phone 268 and any connected application running on the smart phone 268 in combination with the relaxation device 10 can make up a relaxation system.

With reference to FIG. 3, a charging port 272 is provided in the enclosure 12 for charging the relaxation device 10. The charging port 272 is in electrical communication with the processing unit 18 and the power source 20, which can be a rechargeable battery. The at least one processing device can also be provided to control LEDs 274 (only one depicted in FIG. 3) that can be mounted in a light ring 276 positioned between the upper shell 32 and the lower shell 34.

A method of operating the relaxation device 10 will be described with reference to FIG. 7. The process shown in FIG. 7 occurs while controlling the vibrator 14 disposed in the enclosure 12 to induce vibrations of the enclosure 12. The vibrator 14 can be controlled to match the inhalation phase and exhalation phase of a desired breathing pattern where the vibrator 14 is turned on during the inhalation phase and exhalation phase of the desired breathing pattern.

The process in FIG. 7 will be described with reference to measuring the user’s cardiac signals to determine if the detected cardiac signal corresponds to a target signal, and the process could be employed with measuring cardiac signals such as heart rate (HR), heart rate variability (HRV), respiratory sinus arrhythmia (RSA), power and frequency. This correspondence does not need to be an exact match between the detected cardiac signal and the target cardiac signal; instead, the correspondence could be within an acceptable range. At 310, a person’s cardiac signals are detected via the electrodes 246 mounted to the enclosure 12 and the at least one processing device, which can include the processing unit 18 and/or the processor found in the smart phone 268. Typically, the enclosure 12 is placed on the user’s sternum, as shown in FIG. 1, while the user is lying down on her back. As mentioned above, other cardiac metrics such as HR, RSA, power or frequency could also be detected at 310.

At 312, the detected cardiac signals are compared to a target cardiac signal via the at least one processing device, which can include software running on the processing unit 18 and/or the processor found in the smart phone 268. The target cardiac signal can be the person’s greatest HRV, which is associated with the person’s resonance frequency and is associated with the person’s optimal state for relaxation and sleep. If desired, other detected cardiac metrics can be compared to the appropriate target cardiac measurement at 312.

At 314, if the detected cardiac signals correspond to the target cardiac signal, then the process moves to 316 and a scent solution is emitted from the enclosure 12 via a scent release mechanism 16. Emitting the scent solution from the enclosure 12 via the scent release mechanism 16 can include atomizing scent solution 64, 164 drawn from a capillary wick 66, 166 disposed in the replaceable scent capsule 54 or the alternative scent capsule 154 received in the enclosure 12. If, at 314, the detected cardiac signals do not correspond to the target cardiac signal, then the process can return to 310 and detect the person’s cardiac signals via the electrodes 246 mounted to the enclosure 12. In other words, no scent may be emitted, which can indicate to the user that the target cardiac signal has not been achieved.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A scent capsule for use with a relaxation device, the scent capsule comprising:
a capsule housing having a volume between 0.5 and-3.0 mL;
a scent solution in the capsule housing; and
a capillary wick extending from an interior of the capsule housing through a wick opening in the capsule housing to an exterior of the capsule housing.

2. The scent capsule of claim 1, wherein the scent solution has a viscosity below 10 centipoise at 20 degrees C.

3. The relaxation device of claim 1, wherein the scent solution has a specific gravity within 20% of distilled water at 20 degrees C.

4. The scent capsule of claim 1, wherein the capillary wick has a diameter between 3 and 8 mm.

5. The scent capsule of claim 1, wherein a free end of the capillary wick extends less than 2.5 mm from the capsule housing.

6. The scent capsule of claim 1, further comprising a magnetic connector mounted to the capsule housing offset radially from the wick opening.

7. The scent capsule of claim 6, wherein the capsule housing includes a hollow body holding the scent solution and a gasket, which is made from a resilient material that is more resilient than a material from which the hollow body is made, covering the magnetic connector.

8. The scent capsule of claim 7, wherein the hollow body includes a plug opening and a plug received in the plug opening, wherein the plug defines the wick opening and the magnetic connector is sandwiched between the gasket and the plug.

9. The scent capsule of claim 8, wherein the gasket is a portion of a sleeve also covering at least a portion of the hollow body.

10. The scent capsule of claim 9, wherein the sleeve covers an entirety of the hollow body.

11. The scent capsule of claim 9, wherein the sleeve includes a tab extending away from a portion of the hollow body located opposite the wick opening.

12. The scent capsule of claim 7, wherein the hollow body includes a capsule wall, a ledge extending inwardly from the capsule wall and a neck extending away from the ledge, wherein the neck defines the wick opening and the magnetic connector is sandwiched between the gasket and the ledge.

13. The scent capsule of claim 12, wherein the gasket is a portion of a jacket that includes at least one band extending from the gasket.

14. The scent capsule of claim 13, wherein the jacket includes a tab extending away from a portion of the hollow body located opposite the wick opening.

15. The scent capsule of claim 13, wherein the jacket includes a base connected with the band opposite the gasket, the base covering a portion of the hollow body located opposite the wick opening and a tab extending away from a portion of the base.

16. The scent capsule of claim 13, wherein the hollow body includes at least one channel extending inwardly from an outer surface of the hollow body and the at least one band is received in the at least one channel.

17. The scent capsule of claim 16, wherein an outermost surface of the band is flush with the outer surface of the hollow body.

18. The scent capsule of claim 17, wherein the jacket includes a base connected with the band opposite the gasket, the base covering a portion of the hollow body located opposite the wick opening and a tab extending away from a portion of the base.
